(19) United States
(12) Patent Application Publication
(10) Pub. No.: US 2008/0029104 A1
(43) Pub. Date: Feb. 7, 2008

(54) ARM MOVEMENT RESTRAINING AND PERMISSION CORD FOR PATIENTS RECEIVING CARDIAC PACEMAKERS AND SIMILAR IMPLANTS

(75) Inventor: Harold Y. Vanderpool, Galveston, TX (US)

Correspondence Address: ROBERT W STROZIER, P.L.L.C, PO BOX 429, BELLAIRE, TX, 77402-0429, US

(73) Assignee: The Board of Regents of The University of Texas System, Austin, TX (US)

(21) Appl. No.: 11/835,331

(22) Filed: Aug. 7, 2007

Related U.S. Application Data
(60) Provisional application No. 60/836,075, filed on Aug. 7, 2006.

(51) Int. Cl. A61F 5/37

(52) U.S. Cl. 128/878

(57) ABSTRACT

An arm movement restraining and permission cord apparatus is disclosed for patients receiving cardiac pacemakers and similar implants to prevent patients for arm movements that may result in health risk due to leads being pulled out from the heart muscle.

### RELATED APPLICATIONS

This application claims priority to U.S. Provisional Patent Application Ser. No. 60/836,075, filed 7 Aug. 2006 (Aug. 7, 2006).

### BACKGROUND OF THE INVENTION

1. Field of the Invention

The present invention relates to an apparatus for restraining arm motion after surgical implantation of a medical device in the chest of a patient.

More particularly, the present invention relates an apparatus for restraining arm motion after surgical implantation of a medical device in the chest of a patient, where the apparatus includes a waist member, a resilient member and a wrist member. The waist member is adapted to detachable attach a first end of the resilient member to the patient's waist (belt, pant loop, etc.), while the wrist member is adapted to attach a second end of the resilient member to the patient's wrist or forearm.

2. Description of the Related Art

After implant surgery in the chest cavity such as the implantation of a cardiac pacemaker, the patient is instructed to do normal exercises with both arms, but not to raise the patient's left arm above shoulder level (in rare cases, the pacemaker leads are positioned in the patient's right arm and the patient's right arm should not be raised above shoulder level). The patient is then given a traditional sling. However, a sling in no way permits normal motion or restrains the patient from raising his/her left arm above shoulder level.

Thus, there is a need in the art for a simple and easy apparatus that can be used by a patient receiving a chest implant that constrains the patient's left arm from being raised above shoulder level during waking and sleeping hours, yet permit normal arm active.

### SUMMARY OF THE INVENTION

The present invention provides a waist member, a resilient member and a wrist member. The waist member is adapted to detachable constrain a first end of the resilient member to the patient's waist, while the wrist member is adapted to constrain the second end of the resilient member to the patient's wrist or forearm.

### BRIEF DESCRIPTION OF THE DRAWINGS

The invention can be better understood with reference to the following detailed description together with the appended illustrative drawings in which like elements are numbered the same.

FIGS. 1A&B depict side and top views of an embodiment of an apparatus of this application.

FIGS. 2A&B depict side and top views of another embodiment of an apparatus of this application.

FIGS. 3A&B depict side and top views of another embodiment of an apparatus of this application.

FIGS. 4A&B depict side and top views of another embodiment of an apparatus of this application.

FIGS. 5A&B depict top views of another embodiment of an apparatus of this application.

FIGS. 6A-D depict pictures of an embodiment of an apparatus of this invention in operation.

### DETAILED DESCRIPTION OF THE INVENTION

The inventors have found that a simple apparatus can be constructed for better restraint of arm motion after the surgical implantation of a device in a patient's chest such as a cardiac pacemaker, cardioverter defibrillators (ICD), or other similar devices. The apparatus allows a patient near normal and full range of motion of one of the patient's arms (in most cases the right arm and in rare cases the left arm), while greatly reducing the patient's ability to inadvertently perform arm movements with the other arm that could lead to post implantation complications. Generally, the apparatus is adapted to restrict the patient from raising the other arm to a level above shoulder level thereby reducing a potential for pulling the leads or electrodes from or out of the heart muscle. The apparatus can be a unitary structure, a two piece structure or a multi-piece structure, including a waist engaging portion or member and a wrist or fore arm engaging portion or member with a resilient portion or member interconnecting the waist and writ portions or members. Of course, if the apparatus is a unitary construction, then the waist and writ portions can be resilient as well.

The present invention relates broadly to an arm restraint including a waist engaging member or portion, a wrist or forearm engaging member or portion and a resilient chord or band member or portion interconnecting the waist and wrist portions or members, where the apparatus is adapted to permit nearly full normal range of motion while restraining one's ability to raise the constrained arm above shoulder level. The apparatus can be made in different sizes or can be adjustable so that one size fits all. Band adjustment can either by made by manufacturing a set of apparatuses of this invention with different length bands, bands having different elastic properties, and different sizes and types of engaging members to accommodate different patient types.

The present invention is radically different visually and functionally from an arm sling. It is much more protective of patients who have recently received a cardiac implant device. The apparatus of this invention physically reminds patients to avoid habitual/life-long arm movements, or dangerous arm movements while sleeping that can threaten the patient's health and/or damage the recently implanted cardiac device. The apparatus also facilitates arm movements that hasten healthy rehabilitation.

The present invention supplements post-operative advice, care, limitations and boundaries that patients should follow after receiving a cardiac pacemaker or cardioverter defibrillator (ICD). The fact that post-operative advice from renowned institutions vary a great deal with respect to the length of time a patient needs to guard against pulling out their implant leads from their heart muscle (this length of time varies from 1 to 8 weeks), suggests that careful and sustained attention and research have not been given to post-operative care. It is likely that this research vacuum accounts for the continuing issuing of tradition arm slings to such patients that are counterproductive to and undermine the two main and constantly-repeated aims of post-operative care, e.g., arm restraint and health-promoting arm movements. The present apparatus was adapted to be highly protective and useful for all persons who receive cardiac pacemaker and implantable defibrillators (ICDs). It might also prove highly useful for other types of post-surgical patients, e.g., offering arm-hand restraints for patients who have had eye surgery and are warned not to tough their eyes.

Introduction

After receiving a cardiac pacemaker, a patient is normally told, at check out from the hospital, to restrict the movement of one arm, in most cases the left arm, in order to keep from pulling out the wire leads of the pacemaker embedded in the patient's heart muscle. A pacemaker is implanted in the chest area next to a non-dominant arm. Therefore, right-handed persons have the pacemaker placed in the chest area proximate to the left arm, while left-handed persons have the pacemaker implanted in the chest area proximate to the right arm.

Because these leads are attached to the pacemaker that is placed in the chest most often proximate to the patients' left arm, patients are warned that raising that arm above shoulder level or picking up anything weighing more than about 5 pounds for several weeks could pull the leads/electrodes out of the patient's heart muscle, thereby threatening the patient's health and requiring the pacemaker operation to be repeated.

Several weeks of restricted movement enable the leads to become securely attached and embedded in the heart muscle. Similar restrictions are given for other types of implantable cardiac devices. While the length of time for constrained arm motion varies, the length of time generally is between 1 and 8 weeks depending on the hospital where the implantation was performed.

These directives regarding what patients should not do are given to the hundreds of thousands (perhaps millions) of persons each year who receive pacemakers and other types of surgically implanted devices. At the same time, patients are told what they can and should do. They should resume most of their normal, non-strenuous activities with the arm nearest to the surgical site. The Cleveland Clinic states: “You may move your arm normally and do not have to restrict its motion during normal daily activities.” In keeping with such directives, pacemaker recipient are told that it is important to move both arms for the sake of better healing, protection against the loss of muscle strength, maintaining arm agility, and so on.

Problems

The problem with these directives regarding what patients should not and should do is that to assist patients in following these directives, patients are issued a standard arm sling, an arm sling designed specification for broken arms, to wear upon leaving the hospital. However, such arm slings are totally counter productive to the post-operative counsel the patients are.

In fact, the traditional sling, because of its design, keep patients from doing what they were told they should be doing, i.e., normal (but non strenuous) movements of the left arm, and have little or no effect on restricting how high the left arm is raised. The design of a traditional arm sling does not prevent a patient from raising his/her arm above shoulder level while awake or asleep. Thus, traditional arm slings are wholly inadequate to prevent or remind patients not to raise their left arm above shoulder level. This lack of protection from a traditional arm sling, has and does cause patients to have to go back to the hospital to have pulled out leads reinserted into the heart muscle.

Solution

The deficiencies in a traditional arm sling are over come by the apparatus of this invention. In its crude form, the apparatus included several stout rubber bands linked together. One end of linked bands is adapted to be attached to a belt or other attaching site at the waist and the other end is adapted to be attached to a wrist of the arm to be restrained from certain motions. A length of the linked bands is adjusted so that it constantly reminded the wearer about to raise his/her arm and elbow. As the wearer raises his/her arm, the band is pulled taut and begins to resist additional raising, reminding the wearer not to raise the arm above shoulder level. The bands can be designed to that it would be difficult to raise the arm above shoulder level. This reminder from the linked bands is progressive. Thus, the band can be adjusted to (1) start restraining the arm before the elbow reached shoulder height, and (2) become more and more restraining as the arm is raised to shoulder height. Furthermore, the flexibility of the bands keeps the wearer from jolting the arm to a stop, which could possibly jerk out one or both of the electrode leads embedded in the heart muscle.

The apparatus of this invention is directly related to and suited for both of the important directives pacemaker patients are given post-implantation. The resilient band directly allows patients to do what they should be doing (normal and non-strenuous movements of the arm) and is a constant reminder/warning of what they should not do, i.e., risk pulling the pacemaker leads out of the heart muscle by raising the arm above the shoulder.

Confirmation of Inventiveness

The apparatus was to implantation doctors and nurses thought the apparatus was great. All recognized that this idea will contribute to both the safety and faster rehabilitation of patients who receive implantable devices with wire leads in the heart muscle.

Operative Specifics

Although linked rubber bands work, a resilient cord such as a small diameter bungee cord with a wrist loop at one end and a length of Velcro at the other represent alternate embodiments.

The bungee cord would, like rubber bands, both soften and gradually increase reminders to the wearer that the arm is being raised too high, in contrast to possibly dangerous stoppage jerks from a non-stretchable or resilient cords. However, non-resilient cord can be used. The length of Velcro would allow patients of different heights to adjust the cord on their belts or belt loops in keeping with the length of their bodies and arms. The apparatus can be manufactured in different colors for color preference and coordination.

Suitable band material (the entire apparatus can be made out of the same material) include, without limitation, cured and uncured elastomers or other viscoelastic polymers, bungee cord material, polyamides such as nylon, polyacrylates, polyolefins, thermoplastic elastomers, animal hide, cotton rope, cloth, carbon fiber rope, composite rope, metal reinforced composite rope or any other resilient or non-resilient materials or mixtures or combinations thereof.

Suitable engaging material include, without limitation, all the above stated materials for use in the band, metals, resilient or non-resilient fabrics, resilient non-resilient plastics, or any other structural material or mixtures or combinations thereof.

### DETAILED DESCRIPTION OF EMBODIMENTS

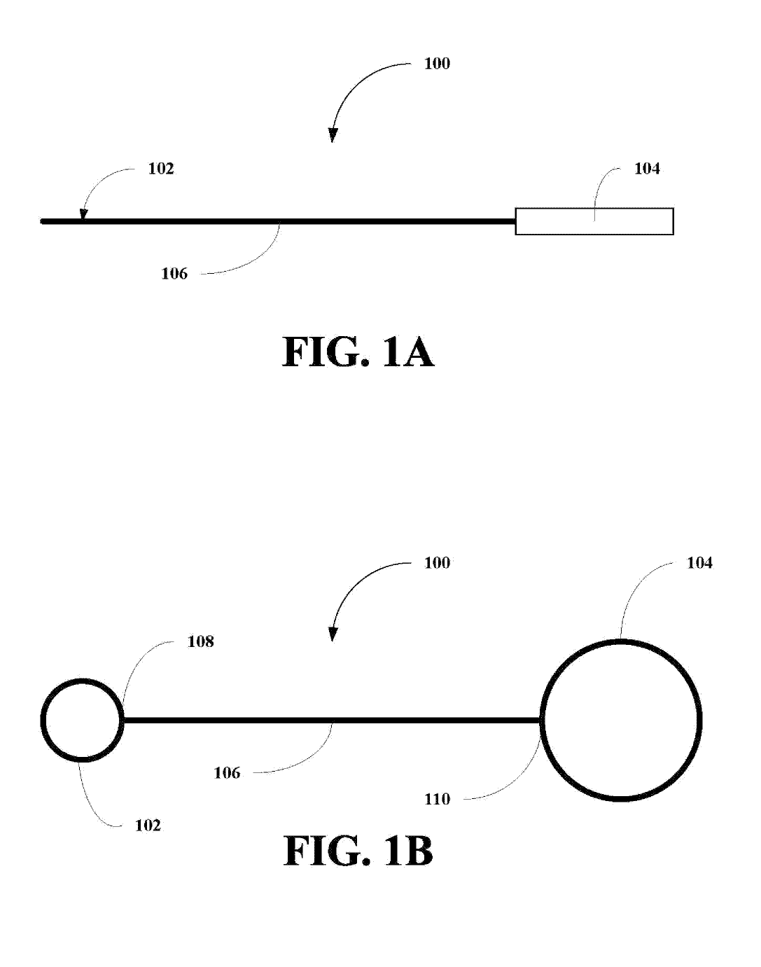

Referring now to FIGS. 1A&B, an apparatus of this invention, generally 100, is shown to be an unitary structure and to include a belt engaging member 102, a wrist or forearm engaging member 104 and an interconnecting band 106. The belt engaging member 102 is adapted to be threaded onto a belt (not shown) to restrain a first end 108 of the band 106 at the patient's waist, chest or leg. The wrist or forearm member 104 is adapted to restrain a second end 110 of the band 106 at the patient's wrist or forearm. The wrist engaging member 104 can be a non-resilient or resilient loop that is passed over a patient's hand onto the wrist or forearm.

In certain embodiment, the band 106 is of a length and has elastic properties (made of a resilient material) adapted to restrain arm raising above shoulder level by gradually increasing resistance as the patient raising his/her arm past towards shoulder level. In these embodiments, the length and elastic properties work together to insure that motion is not brought to a sudden stop, but make the process of raising one's arm more and more difficult. In other embodiments, the band 106 is non or substantially non-elastic (made of a non-resilient material) causing a sudden stop of motion when the patient attempts to raise his/her arm too high, with the length adjusted such that the patient cannot raise his/her arm past a certain height. The apparatus 100 is adapted to be worn all the time. Of course, at night, the patient would have to wear a belt around the waste, chest or leg so that the apparatus 100 would continue to restrain any arm raising motion during sleep.

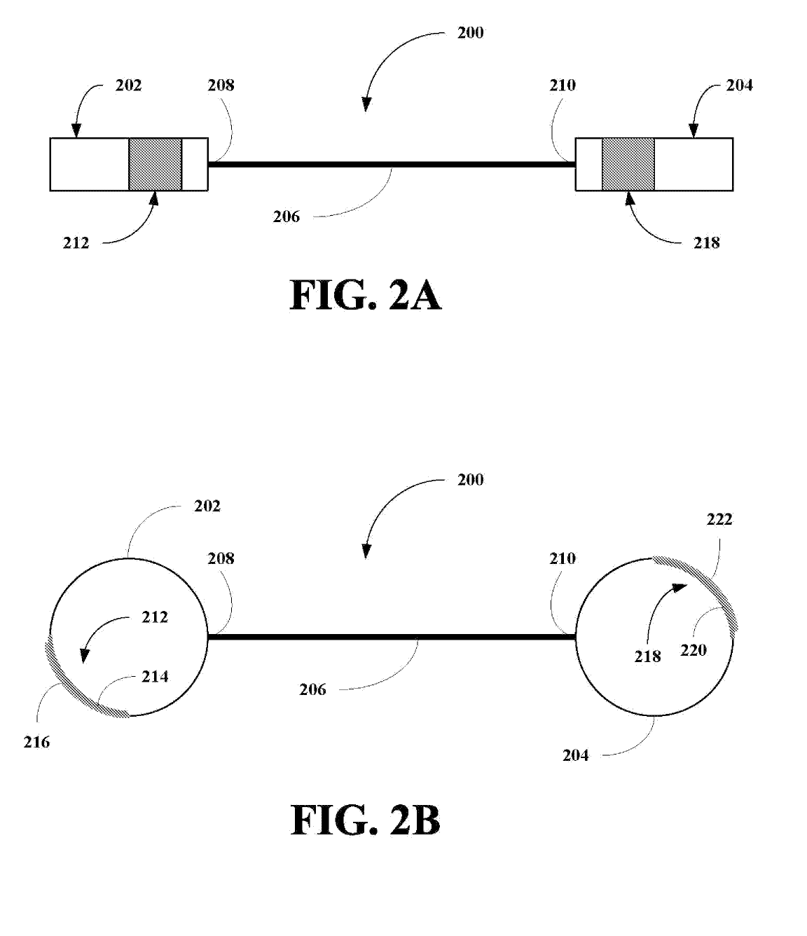

Referring now to FIGS. 2A&B, another apparatus of the invention, generally 200, is shown to include a first engaging member 202, a second engaging member 204 and an interconnecting band 206, where the first engaging member 202 is adapted to engage a belt positioned around a patient's waist, chest or leg and the second engaging member 204 is adapted to engage a patient's wrist or fore arm. The first member 202 is adapted to restrain a first end 208 of the band 206 at the patient's waist, chest or leg. The second member 204 is adapted to restrain a second end 210 of the band 206 at the patient's wrist or forearm. The first engaging member 202 includes a hook-and-loop connection 212 having a loop part 214 and a hook part 216 so that the member 202 can be easily and quickly detachable connected to the belt (not shown) without having to remove the belt. The second engaging member 204 also includes a hook-and-loop connection 218 having a loop part 220 and a hook part 222 so that the member 204 can be easily and quickly detachable connected about the patient's wrist or forearm.

Again, in certain embodiment, the band 206 is of a length and has elastic properties (made of a resilient material) adapted to restrain arm raising above shoulder level by gradually increasing resistance as the patient raising his/her arm past towards shoulder level. In these embodiments, the length and elastic properties work together to insure that motion is not brought to a sudden stop, but make the process of raising one's arm more and more difficult. In other embodiments, the band 206 is non or substantially non-elastic (made of a non-resilient material) causing a sudden stop of motion when the patient attempts to raise his/her arm too high, with the length adjusted such that the patient cannot raise his/her arm past a certain height. The apparatus 200 is adapted to be worn all the time. Of course, at night, the patient would have to wear a belt around the waste, chest or leg so that the apparatus 200 would continue to restrain any arm raising motion during sleep.

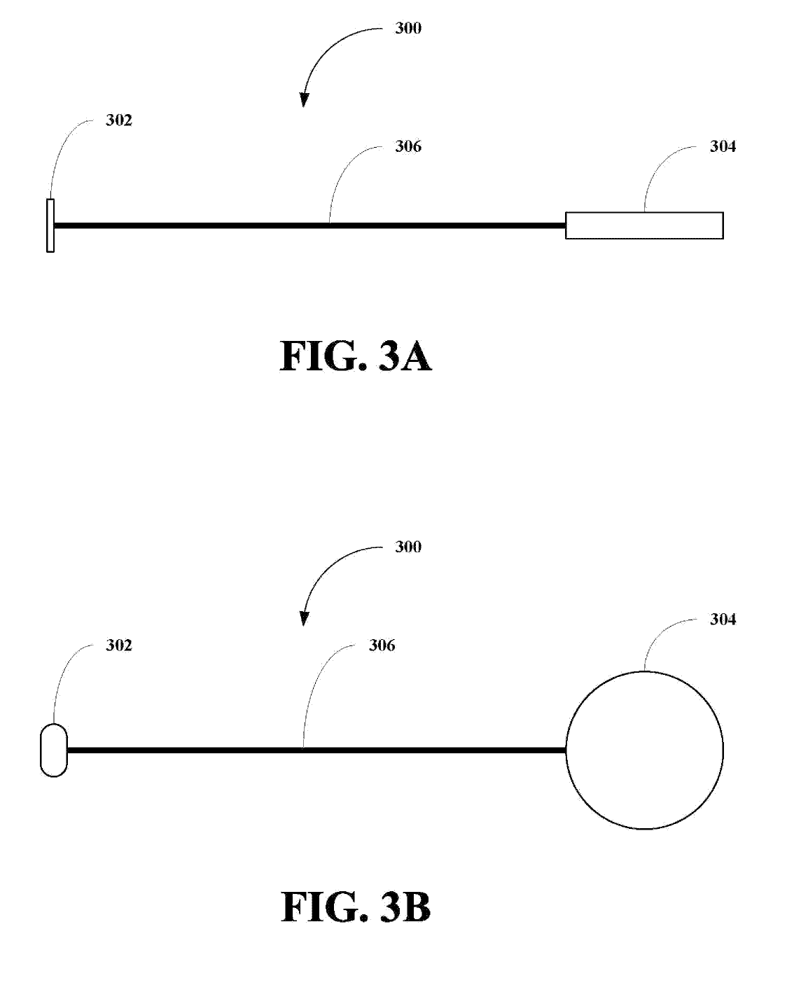

Referring now to FIGS. 3A&B, another apparatus of the invention, generally 300, is shown to include a first engaging member 302, a second engaging member 304 and an interconnecting band 306, where the first engaging member 302 is adapted to engage a belt positioned around a patient's waist, chest or leg and the second engaging member 304 is adapted to engage a patient's wrist or fore arm. The first engaging member 302 comprises a belt loop adapted to be threaded onto a belt (not shown) to restrain a first end 308 of the band 306 at the patient's waist, chest or leg. The second engaging member 304 comprises a resilient band adapted to restrain a second end 310 of the band 306 at the patient's wrist or forearm. The wrist engaging member 304 can be a non-resilient or resilient loop that is passed over a patient's hand onto the wrist or forearm.

Again, in certain embodiment, the band 306 is of a length and has elastic properties (made of a resilient material) adapted to restrain arm raising above shoulder level by gradually increasing resistance as the patient raising his/her arm past towards shoulder level. In these embodiments, the length and elastic properties work together to insure that motion is not brought to a sudden stop, but make the process of raising one's arm more and more difficult. In other embodiments, the band 306 is non or substantially non-elastic (made of a non-resilient material) causing a sudden stop of motion when the patient attempts to raise his/her arm too high, with the length adjusted such that the patient cannot raise his/her arm past a certain height. The apparatus 300 is adapted to be worn all the time. Of course, at night, the patient would have to wear a belt around the waste, chest or leg so that the apparatus 300 would continue to restrain any arm raising motion during sleep.

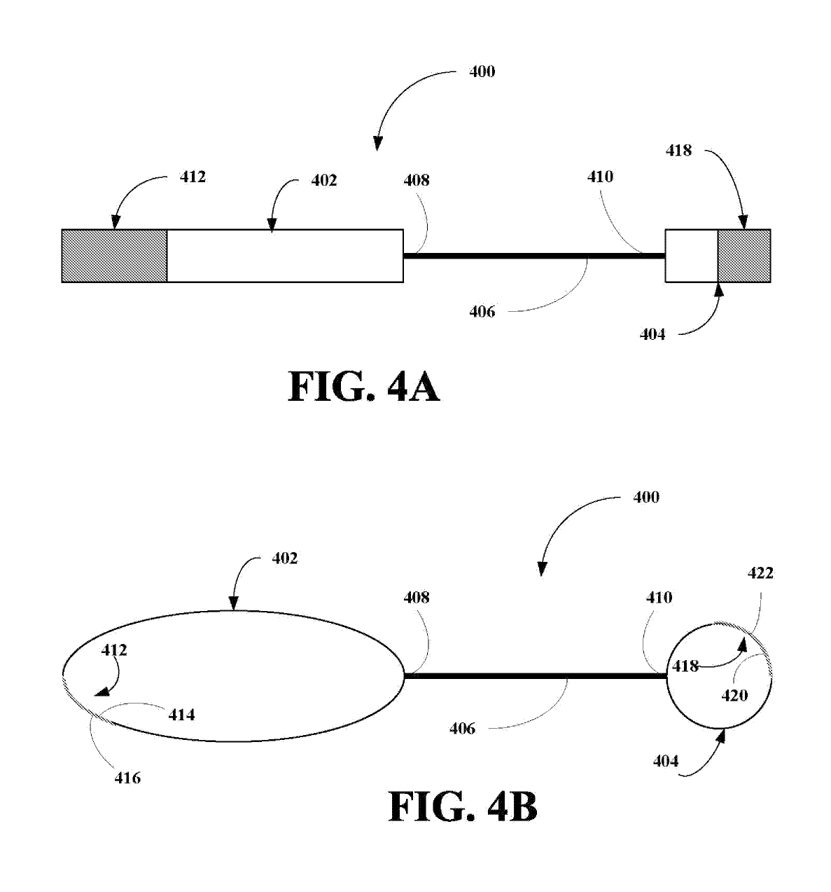

Referring now to FIGS. 4A&B, another apparatus of the invention, generally 400, is shown to include a first engaging member 402, a second engaging member 404 and an interconnecting band 406, where the first engaging member 402 is adapted to engage by wrapping around a patient's waist, chest or leg and the second engaging member 404 is adapted to engage a patient's wrist or fore arm. The first member 402 is adapted to restrain a first end 408 of the band 406 at the patient's waist, chest or leg. The second member 404 is adapted to restrain a second end 410 of the band 406 at the patient's wrist or forearm. The first engaging member 402 includes a hook-and-loop connection 412 having a loop part 414 and a hook part 416 so that the member 402 can be easily and quickly detachable connected to the belt (not shown) without having to remove the belt. The second engaging member 404 also includes a hook-and-loop connection 418 having a loop part 420 and a hook part 422 so that the member 404 can be easily and quickly detachable connected about the patient's wrist or forearm.

Again, in certain embodiment, the band 406 is of a length and has elastic properties (made of a resilient material) adapted to restrain arm raising above shoulder level by gradually increasing resistance as the patient raising his/her arm past towards shoulder level. In these embodiments, the length and elastic properties work together to insure that motion is not brought to a sudden stop, but make the process of raising one's arm more and more difficult. In other embodiments, the band 406 is non or substantially non-elastic (made of a non-resilient material) causing a sudden stop of motion when the patient attempts to raise his/her arm too high, with the length adjusted such that the patient cannot raise his/her arm past a certain height. The apparatus 400 is adapted to be worn all the time. Of course, at night, the patient would have to wear a belt around the waste, chest or leg so that the apparatus 400 would continue to restrain any arm raising motion during sleep.

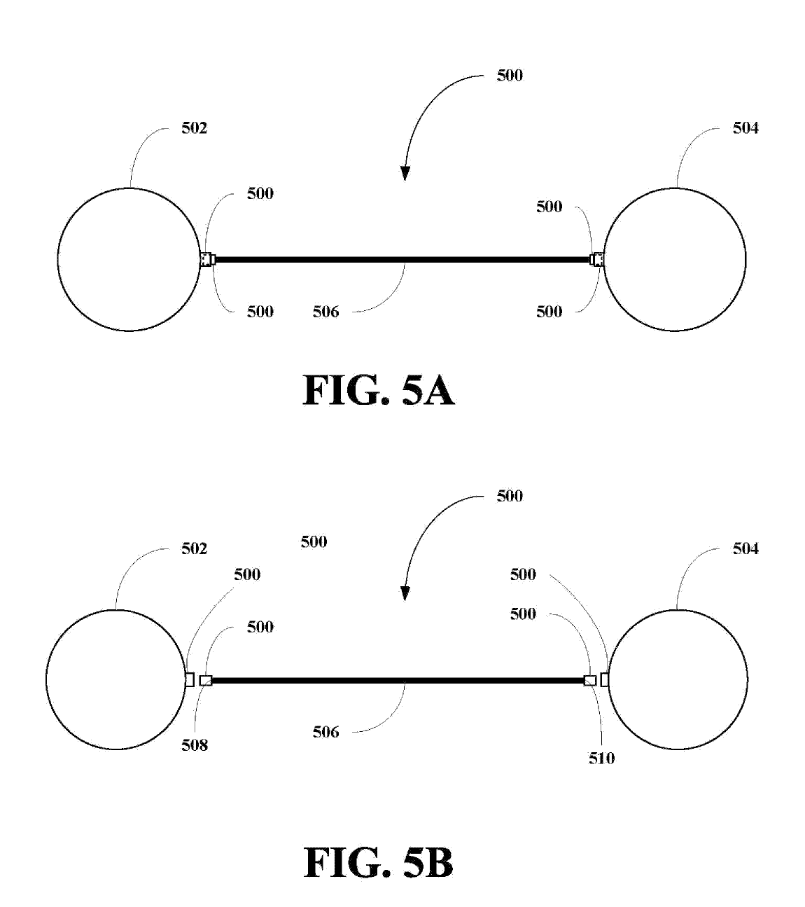

Referring now to FIGS. 5A&B, another apparatus of the invention, generally 500, is shown to include a first engaging member 502, a second engaging member 504 and an interconnecting band 506, where the first engaging member 502 is adapted to engage by wrapping around a patient's waist, chest or leg and the second engaging member 504 is adapted to engage a patient's wrist or fore arm. The first member 502 is adapted to restrain a first end 508 of the band 506 at the patient's waist, chest or leg. The second member 504 is adapted to restrain a second end 510 of the band 506 at the patient's wrist or forearm. The first engaging member 502 includes a connector 512 and the second engaging member 504 includes a connector 514. The band 506 includes a first end connector 516 and a second end connector 518. The first engaging member connector 512 and the second engaging member connector 514 are adapted to lockingly and detachably engage the first or second end connectors 516 or 518 of the band 506 so that the band 506 can be easily and quickly detachable from the engaging member 502 and 504. The connectors 512, 514, 516 and 518 can be any connector known or to be invented that can be from a locking, detachable connection such as a look-and-loop connection, a magnet connection, a magnetic strip connection, a snap connection, a threaded connection, a shaft and pin connection, a leash connection or any other connection capable of connecting the engaging members 502 and 504 to the band 506. The engaging members 502 and 504 can be any of the engaging member including the engaging member described above. The wrist engaging member 504 can be a non-resilient or resilient loop that is passed over a patient's hand onto the wrist or forearm.

Again, in certain embodiment, the band 506 is of a length and has elastic properties (made of a resilient material) adapted to restrain arm raising above shoulder level by gradually increasing resistance as the patient raising his/her arm past towards shoulder level. In these embodiments, the length and elastic properties work together to insure that motion is not brought to a sudden stop, but make the process of raising one's arm more and more difficult. In other embodiments, the band 506 is non or substantially non-elastic (made of a non-resilient material) causing a sudden stop of motion when the patient attempts to raise his/her arm too high, with the length adjusted such that the patient cannot raise his/her arm past a certain height. The apparatus 500 is adapted to be worn all the time. Of course, at night, the patient would have to wear a belt around the waste, chest or leg so that the apparatus 500 would continue to restrain any arm raising motion during sleep.

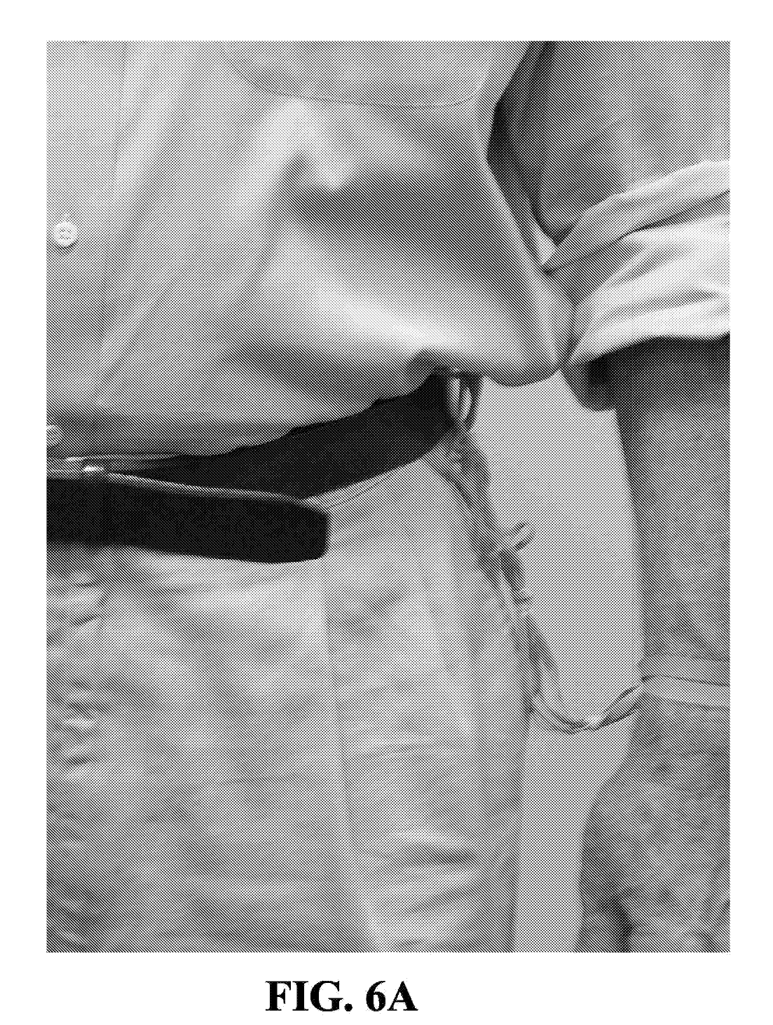

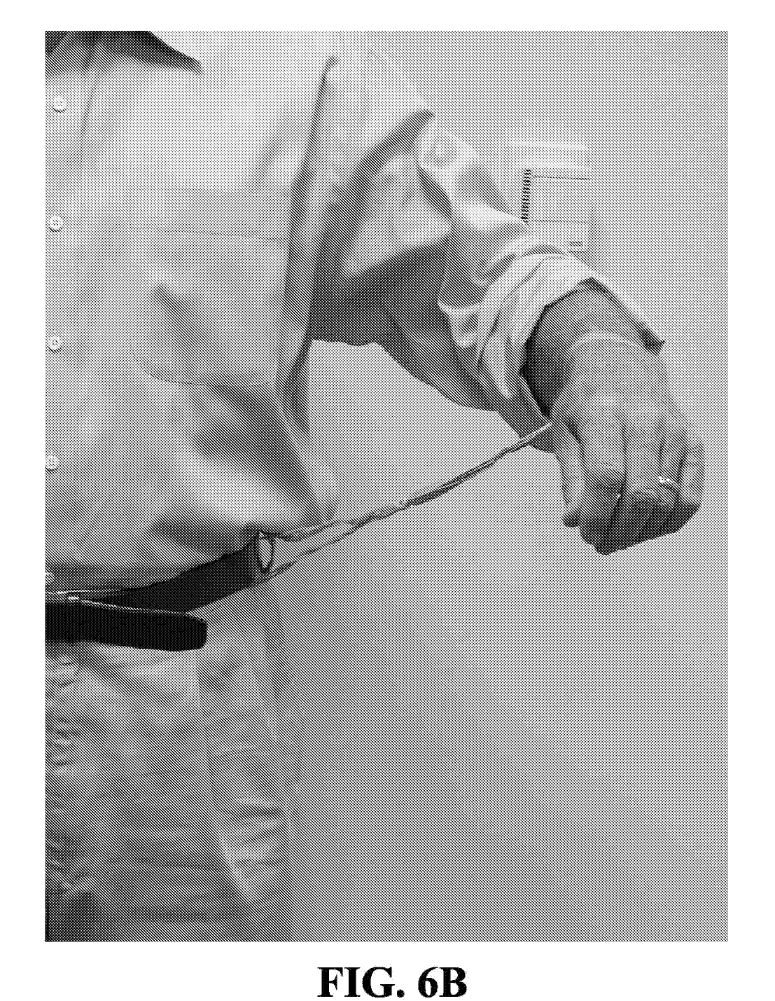

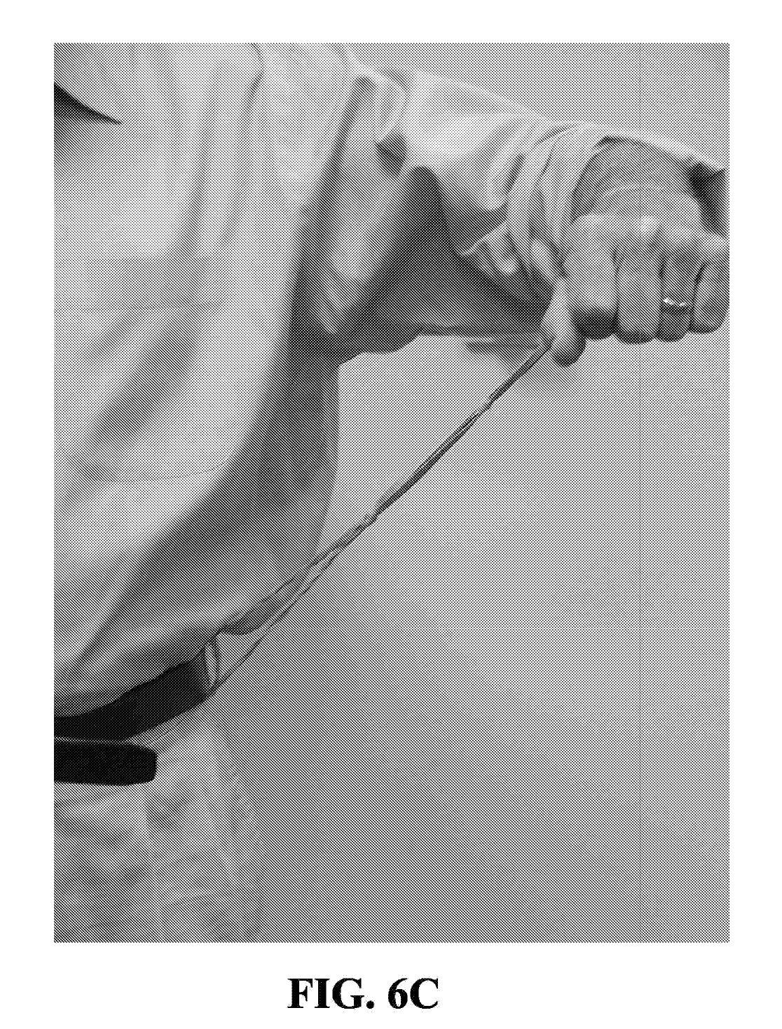

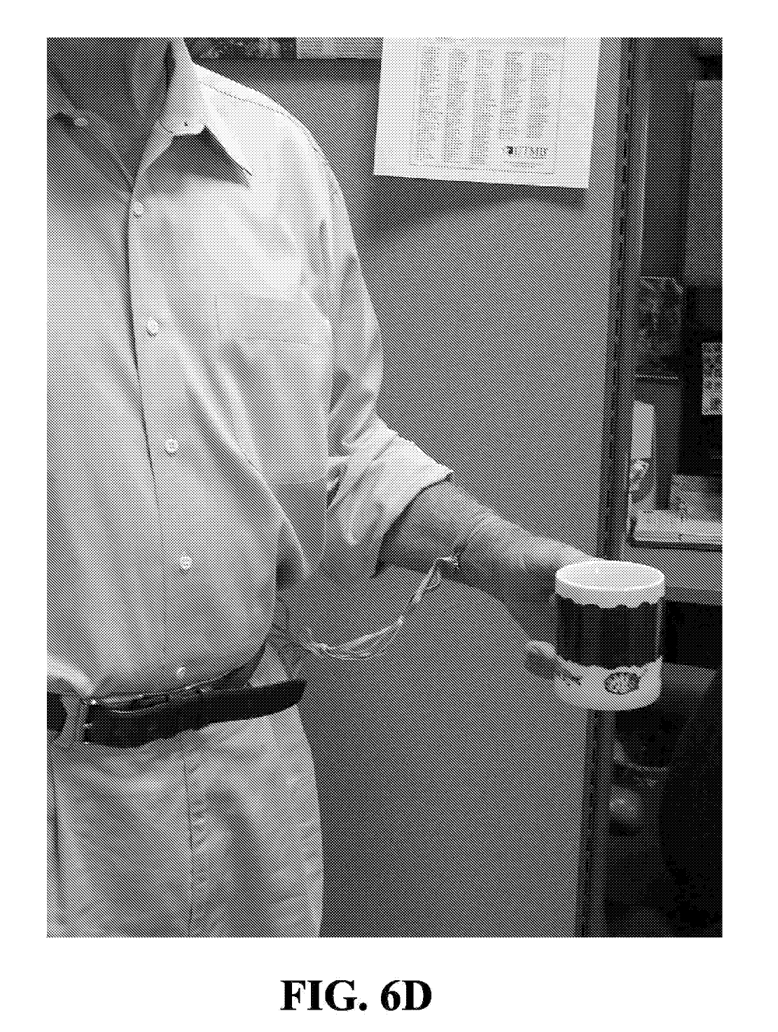

Referring now to FIGS. 6A-D, photographs of another apparatus of the invention are shown demonstrating the use, permitted range of motion and restraining range of motion.

All references cited herein are incorporated by reference. Although the invention has been disclosed with reference to its preferred embodiments, from reading this description those of skill in the art may appreciate changes and modification that may be made which do not depart from the scope and spirit of the invention as described above and claimed hereafter.

I claim:

1. An arm restrain apparatus for patients receiving an implant cardiac device comprising:
a first engaging member,
a second engaging member, and
a band interposed therebetween,
where the first engaging member is adapted to engage the patient's waist, chest or leg, the second engaging member is adapted to engage the patient's wrist or forearm of the arm nearest cardiac device's leads and a length of the band is adapted to prevent or reduce movement of that arm above shoulder height.

2. The apparatus of claim 1, wherein the band is resilient and increases a resistance to movement as that arm is move towards shoulder level.

3. The apparatus of claim 1, wherein the band is non-resilient and stops movement of that arm at should level.

4. The apparatus of claim 1, wherein:
the first engaging member includes a first connector,
the second engaging member includes a second connector, and
the band include a first end connector and a second end connector,
where each engaging member connector is adapted to engage one of the band connectors for form locking, detachable connections.

5. The apparatus of claim 1, wherein the first engaging member includes a look-and-loop connection for easy and quick detachment of the engaging member.

6. The apparatus of claim 1, wherein the second engaging member includes a look-and-loop connection for easy and quick detachment of the engaging member.

7. The apparatus of claim 1, wherein the first engaging member includes a look-and-loop connection for easy and quick detachment of the engaging member and the second engaging member includes a look-and-loop connection for easy and quick detachment of the engaging member.

8. The apparatus of claim 1, wherein the first engaging member comprises a belt engaging member adapted to engage a belt positioned around the patient's waist, chest or leg.

9. The apparatus of claim 1, wherein the second engaging member comprises a resilient loop adapted to slide over the patient's hand onto the patient's wrist or forearm.

10. An arm restrain apparatus for patients receiving an implant cardiac device comprising:
a first engaging member including a look-and-loop connection for easy and quick detachment of the first engaging member,
a second engaging member including a look-and-loop connection for easy and quick detachment of the second engaging member, and
a band interposed therebetween,
where the first engaging member is adapted to engage the patient's waist, chest or leg, the second engaging member is adapted to engage the patient's wrist or forearm of the arm nearest cardiac device's leads and a length of the band is adapted to prevent or reduce movement of that arm above shoulder height.

11. The apparatus of claim 10, wherein the band is resilient and increases a resistance to movement as that arm is move towards shoulder level.

12. The apparatus of claim 10, wherein the band is non-resilient and stops movement of that arm at should level.

13. The apparatus of claim 10, wherein:
the first engaging member includes a first connector,
the second engaging member includes a second connector, and
the band include a first end connector and a second end connector,
where each engaging member connector is adapted to engage one of the band connectors for form locking, detachable connections.

14. The apparatus of claim 10, wherein the first engaging member comprises a belt engaging member adapted to engage a belt positioned around the patient's waist, chest or leg.

15. The apparatus of claim 10, wherein the second engaging member comprises a resilient loop adapted to slide over the patient's hand onto the patient's wrist or forearm.

16. An arm restrain apparatus for patients receiving an implant cardiac device comprising:
a first engaging member including a first connector,
a second engaging member including a second connector, and
a band including a first end connector and a second end connector interposed therebetween,
where the first engaging member is adapted to engage the patient's waist, chest or leg, the second engaging member is adapted to engage the patient's wrist or forearm of the arm nearest cardiac device's leads and a length of the band is adapted to prevent or reduce movement of that arm above shoulder height.

17. The apparatus of claim 16, wherein the band is resilient and increases a resistance to movement as that arm is move towards shoulder level.

18. The apparatus of claim 16, wherein the band is non-resilient and stops movement of that arm at should level.

19. The apparatus of claim 16, wherein the first engaging member includes a look-and-loop connection for easy and quick detachment of the engaging member.

20. The apparatus of claim 16, wherein the second engaging member includes a look-and-loop connection for easy and quick detachment of the engaging member.

21. The apparatus of claim 16, wherein the first engaging member includes a look-and-loop connection for easy and quick detachment of the engaging member and the second engaging member includes a look-and-loop connection for easy and quick detachment of the engaging member.

22. The apparatus of claim 16, wherein the first engaging member comprises a belt engaging member adapted to engage a belt positioned around the patient's waist, chest or leg.

23. The apparatus of claim 16, wherein the second engaging member comprises a resilient loop adapted to slide over the patient's hand onto the patient's wrist or forearm.
